(19) United States
(12) Patent Application Publication
(10) Pub. No.: US 2002/0147462 A1
(43) Pub. Date: Oct. 10, 2002

(54) Bronchial occlusion method and apparatus

(75) Inventors: Eric A. Mair, Rockville, MD (US); Daniel L. Hedgpeth, Raleigh, NC (US); Melanie A. Vander Klok, Raleigh, NC (US); Jeffrey G. Clark, Raleigh, NC (US); Anthony J. Sherbondy, Raleigh, NC (US)

Correspondence Address: OLIFF & BERRIDGE, PLC, P.O. BOX 19928, ALEXANDRIA, VA, 22320, US

(73) Assignee: Closure Medical Corporation, Raleigh, NC

(21) Appl. No.: 09/949,644

(22) Filed: Sep. 12, 2001

Related U.S. Application Data
(60) Provisional application No. 60/287,402, filed on May 1, 2001.
(60) Provisional application No. 60/231,569, filed on Sep. 11, 2000.

(51) Int. Cl. A61M 37/00; A62B 7/00; A62B 9/00; A62B 18/00; A61D 1/00; A61B 17/00

(52) U.S. Cl. 606/213; 128/887; 128/200.24; 604/82

(57) ABSTRACT

Achieving lung volume reduction includes occluding a lumen of a bronchial tube of a lung to prevent air flow to at least a region of the lung. Bronchial occluders such as polymerizable materials and mechanical devices, such as sutures, staples, clips, clamps, foam, balloons, umbrellas and ball bearings are provided for occluding a bronchial tube. Methods include mixing thickeners or foaming agents with polymerizable compositions and introducing the mixture into a lumen of a bronchial tube. Mechanisms for mixing components and delivering the mixture to a lumen of a bronchial tube are also provided.

### BACKGROUND OF THE INVENTION

1. Field of the Invention

The invention relates to the use of bronchial occluders, including monomer and polymer adhesive compositions. More particularly, the present invention relates to the use of such occluders and compositions to achieve lung volume reduction.

2. State of the Art

Lung volume reduction surgery (LVRS) is the only generally accepted surgical means of reducing lung volume in patients with chronic pulmonary disorders, such as emphysema. LVRS reduces the size of a damaged lung by removing areas of poorly functioning lung tissue, allowing the remaining healthy, or less damaged, lung tissue to function better. However, LVRS requires a thoracotomy, which results in pain and added risks. Also, some patients even experience a worsening of lung function after undergoing LVRS.

In LVRS, a surgeon identifies regions of the lung that are most severely affected by the disease or chronic disorder, such as emphysema, and performs limited resections of these regions. This requires suturing or stapling of the lung to close the surgical wound. The surgeon may opt to close the wound using fibrin glue or a cyanoacrylate medical adhesive, such as that disclosed in U.S. Pat. Nos. 5,928,611 and 5,328,687 to Leung et al., to appose surgically incised tissues. However, lung resection is often complicated by prolonged air leaks leading to lengthy hospital stays and often requiring chest tube placement to allow for drainage.

Various procedures have been used to treat fistulae and/or bronchopleural fistulae without achieving lung volume reduction. See S. Okada et al., “Emergent Bronchofiberoptic bronchial occlusion for intractable pneumothorax with severe emphysema,” Jpn J Thorac Cardiovasc Surg, 46(11), November 1998, pp. 1078-81; C. Jones et al., “Closure of a benign broncho-oesophageal fistula by endoscopic injection of bovine collagen, cyanoacrylate glue and gelfoam,” Aust N Z J Surg., 66(1), January 1996, pp. 53-55; J. Eng et al., “Successful closure of bronchopleural fistula with adhesive tissue [sic],” Scand J Thorac Cardiovasc Surg, 24(2), 1990, pp. 157-59; J. W. Menard et al., “Endoscopic closure of bronchopleural fistulas using a tissue adhesive,” Am J Surg, 155(3), March 1988, pp. 415-16; and G. Inaspettato et al., “Endoscopic treatment of bronchopleural fistulas using n-butyl-2-cyanoacrylate,” Surg Laparosc Endosc, 4(1), February 1994, pp. 62-64. Okada et al. discloses the use of fibrin glue and mesh to plug a bronchopleural fistula in a patient also suffering from emphysema. The procedure does not result in lung volume reduction, but rather treats the fistula at the site of the fistula. This requires the surgeon to probe deeper into the lung, and further provides no beneficial effect for the treatment of the patient's emphysema.

### SUMMARY OF THE INVENTION

The present invention provides a method to achieve lung volume reduction. The present invention may be surgically non-invasive or may be used in conjunction with an invasive surgical procedure. The present invention provides a method of using various bronchial occluders including adhesive compositions such as, but not limited to, adhesive compositions containing polymerizable monomers, such as 1,1-disubstituted ethylene monomers, to block air flow to damaged lung tissue.

The present invention provides a method of achieving lung volume reduction, comprising occluding a lumen of a bronchial tube of a lung to prevent air flow to at least a region of the lung.

The present invention also provides a method of occluding a lumen of a bronchial tube including introducing at least one bronchial occluder into the lumen to occlude the lumen. Such bronchial occluders include, but are not limited to, solid pulmonary occlusive devices, such as metallic devices, for example ball bearings, clips, clamps and sutures, polymers, for example polymerizable materials, preformed solid polymerics, deposited solutions, viscous liquids, and various combinations of the above.

The present invention also provides a method of achieving lung volume reduction comprising mixing a thickener or filler with a biocompatible composition comprising at least one monomer that forms a medically acceptable polymer to form a mixture, and introducing the mixture into a lumen of a bronchial tube of a lung to prevent air flow to at least a region of the lung.

The present invention also provides an apparatus for achieving lung volume reduction comprising a means for mixing at least one component with a biocompatible composition comprising at least one monomer that forms a medically acceptable polymer to form a mixture, and a means for introducing the mixture into a lumen of a bronchial tube of a lung to prevent air flow to at least a region of the lung.

The present invention also provides an apparatus for mixing components comprising at least a first and second syringe removably attached to a mixing valve having at least a coupling point to connect each of said first and second syringes to said mixing valve; and at least a first and second plunger movable within each syringe, wherein the components are moved back and forth between the syringes by alternately depressing the plungers to mix the components prior to extruding the mixed components.

The present invention may be used to treat patients with lung disease, chronic pulmonary disorders, pneumothorax, fistulae, and bronchopleural fistulae. For example, when a bronchial tube leading to an area of emphysemic or diseased or damaged lung is occluded, that area of lung distal to the occlusion will subsequently deflate, leading to atelectasis of lung tissue distal to the occlusion. Thus aspiration or removal of lung tissue, with the associated difficulty, complexity, expense and risk, is not required. The results of the process lead to space for healthy lung tissue to expand and inflate, and in the case of a fistula, prevents air from escaping from the lung into the pleural space or chest cavity.

In the event of a traumatic or disease-induced injury, the present invention may also be used to prevent blood or other fluid in a damaged lung or portion thereof from spilling over into an undamaged lung or portion thereof, thus preventing such complications as hemorrhagic asphyxia. The method of the present invention has particular application to lacerated, incised or punctured lung tissue, for medical or military use. The method may also be used to stop air leaks from a damaged lung. Elimination of airflow from the injured lung into the pleural space or chest cavity may reduce the need for chest tube placement and lengthy hospital stays.

The invention further comprises kits containing mechanical and chemical components of the invention as described herein, preferably in optionally sterilized containers, and more preferably including instructions for practice of methods of the invention.

### BRIEF DESCRIPTION OF THE DRAWINGS

Exemplary embodiments of this invention will be described in detail, with reference to the following drawing figure, in which:

FIG. 1 is a view of a mixing apparatus of the present invention;

FIG. 2 is a schematic view of a method of the present invention using an occlusion balloon of the present invention and a polymerizable material;

FIG. 3 is a schematic view of a second method of the present invention using an inverted spherical occlusion balloon of the present invention and a polymerizable material;

FIG. 4 is a schematic view of a method of the present invention using an occlusion umbrella of the present invention and a polymerizable material; and

FIG. 5 is a perspective view of an embodiment of the occlusion umbrella showing a “snow-flake” pattern of the umbrella ribs and protrusions.

FIG. 6 is a perspective view of an occlusion umbrella of the present invention having claws.

### DETAILED DESCRIPTION OF PREFERRED EMBODIMENTS

The present invention provides a method of achieving lung volume reduction, comprising occluding a lumen of a bronchial tube of a lung to prevent air flow to at least a region of the lung.

For the purposes of this invention, the term “bronchial tube” means a bronchus or any of its branches, including bronchia, bronchioles, or alveoli.

For the purposes of this invention, the term “occlude” or “occlusion” means to form a plug in, or to close off and obstruct, a passageway, particularly with reference to blocking or substantially blocking air flow through a bronchial tube.

For the purposes of this invention, the term “bronchial occluder(s)” means any device, substance or material used to occlude a bronchial tube. Examples of bronchial occluders are polymerizable monomers and adhesives such as cyanoacrylate; solid or hollow devices, such as ball bearings, catheterization-type balloons, small umbrella-shaped devices (further described below and hereinafter referred to as “umbrellas”), iris diaphragms such as the WL Gore HELEXTM septal occluder, sutures, staples or clamps; and various combinations of bronchial occluders, such as a solid or hollow device inserted in a bronchial tube in combination with a cyanoacrylate adhesive.

For the purposes of this invention, the term “lumen” refers to the inner open space or cavity of a bronchial tube.

For the purposes of this invention, the term “lung volume reduction” means the result or procedure to reduce the gross volume or capacity of a lung or lungs.

Methods of the present invention may, in embodiments, be performed via catheter delivery, such as performed through the endotracheal tube, and using bronchoscopy and/or bronchoscopes, such as rigid or fiberoptic bronchoscopes, for direct visualization. Use of radio-opaque agents in or in conjunction with occluders of the invention facilitates such visualization, e.g., by fluoroscopy. The methods of the present invention may also, in embodiments, be performed via laparoscopy. The methods of the present invention may also, in embodiments, be performed in conjunction with open surgery, such as a thoracotomy. However, a particular benefit of embodiments of the present invention is the elimination of the need for invasive procedures.

Any of a variety of materials may be used to occlude a bronchial tube and prevent air from flowing or substantially diminish air flow into the obstructed region. For example, polymerizable monomers, medical adhesives, preformed porous, solid or hollow bodies, deposited solutions, viscous liquids, semi-solids, soft materials or solids, ball bearings, balloons, umbrellas and combinations of the above may be used. Bronchial occluders, such as ball bearings, balloons, umbrellas and preformed bodies are preferably shaped to allow them to be wedged and/or adhered in a lumen of a bronchial tube. Any nontoxic material suitable as a medical device that could adequately restrict or prevent airflow, and preferably also microorganism transit, for a sufficient time could be used.

Bronchial occluders may be placed into a bronchial tube using various endoscopic and bronchoscopic visualization techniques. Forceps, catheters, or other suitable instruments, may be used to place a bronchial occluder into a suitable position within the bronchial tube. In the case of a mechanical device, viscous liquid, deposited solution, polymerizable monomer, adhesive, etc., various catheters, such as a single or dual lumen catheter, and other endotracheal applicators may be used.

The location of the occlusion and/or the placement of the bronchial occluder may vary depending on the location of the lung injury, damage or disease. In embodiments, it may be preferable to place the selected bronchial occluder in or at a bifurcation or branching of the lung to further secure the bronchial occluder, which may assist the bronchial occluder in resisting displacement and dislodgment forces. Thus for example complete occlusion of a bronchus and subsegmental bronchi may be helpful to ensure good long term occlusion. Occlusion in a way that fills and occludes multiple bronchial branches at one or more areas of bifurcation, for example by solidification of a liquid, gel or paste, is particularly advantageous. Adhesion to a mucous-coated bronchial wall may often be imperfect; such an approach creates a strong mechanical bond between the occluder and lung, thereby avoiding slippage and leakage.

Preferably, the bronchial occluders and/or packaging therefor are sterilized to limit risks of infection. Preferably, the bronchial occluders have a Sterility Assurance Level (SAL) of from 10⁻³ to 10⁻⁶. When sterilized, the bronchial occluders may be sterilized by any suitable sterilization procedure. Any of the above-mentioned bronchial occluders, whether sterilized or not, may be used in combination with (e.g., coated or admixed with) various bioactive materials.

Suitable bioactive materials include, but are not limited to, medicaments such as antibiotics, antimicrobials, antiseptics, antibacterials, bacteriocins, bacteriostats, disinfectants, steroids, anesthetics, fungicides, anti-inflammatory agents, antibacterial agents, antiviral agents, antitumor agents (including radioactive and chemotherapeutic agents), growth promoting substances, other desired active agents to assist in preventing the spread of infection and/or to deliver a specified medicinal agent to the lung tissue, or mixtures thereof. Such compounds include, but are not limited to, acetic acid, aluminum acetate, bacitracin, bacitracin zinc, benzalkonium chloride, benzethonium chloride, betadine, calcium chloroplatinate, certrimide, cloramine T, chlorhexidine phosphanilate, chlorhexidine, chlorhexidine sulfate, chloropenidine, chloroplatinatic acid, ciprofloxacin, clindamycin, clioquinol, cysostaphin, gentamicin sulfate, hydrogen peroxide, iodinated polyvinylidone, iodine, iodophor, minocycline, mupirocin, neomycin, neomycin sulfate, nitrofurazone, non-onynol 9, potassium permanganate, penicillin, polymycin, polymycin B, polymyxin, polymyxin B sulfate, polyvinylpyrrolidone iodine, povidone iodine, 8-hydroxyquinoline, quinolone thioureas, rifampin, rifamycin, silver acetate, silver benzoate, silver carbonate, silver chloride, silver citrate, silver iodide, silver nitrate, silver oxide, silver sulfate, sodium chloroplatinate, sodium hypochlorite, sphingolipids, tetracycline, zinc oxide, salts of sulfadiazine (such as silver, sodium, and zinc), and mixtures thereof. Preferable bioactive materials are USP approved, more preferably USP monographed.

Additionally, it is preferable that the bronchial occluders do not biodegrade for at least a period of 1 month, 1 year, 2 years, 3 years or more. In some situations, a bronchial occluder that never biodegrades may be preferable. Preferably, the above-mentioned bronchial occluders provide a permanent or at least semi-permanent occlusion of the bronchial tube. For the purposes of the present invention, the term “permanent” means an occluder that will not substantially biodegrade for at least 2 years. For the purposes of the present invention, the term “semi-permanent” means an occluder that may be removed or that will biodegrade in some period of time, preferably within 1 month to 2 years. For example, for the treatment of chronic disorders, such as emphysema, the bronchial occluder would preferably be permanent. However, in some situations, a semi-permanent occlusion may be preferable so that the occlusion may be removed or allowed to biodegrade, for example, after the lung has healed from a surgical or traumatic wound. Preferably, the bronchial occluder is permanently resident or at least semi-permanently resident in the bronchial tube after introduction into or onto the bronchial tube. The time period in which the bronchial occluder is resident in or on the bronchial tube may be fixed or controlled by one skilled in the art in light of the disclosure of this specification.

According to embodiments of the present invention, solid, liquid, gel, paste or the like pulmonary occlusive devices, such as metallic devices, for example ball bearings, clips, clamps and sutures, polymers, polymerizable materials, preformed solid polymerics, deposited solutions, viscous liquids, and various combinations of the above, including but not limited to combinations of pre-formed and in situ-formed occlusive devices, may be used to occlude a region of affected lung tissue. In embodiments, a preformed physical bronchial occluder, such as an umbrella, balloon, foam or ball bearing may be used. Polymerizable materials may be, for example, monomers and monomer systems, cyanoacrylate, acrylate, epoxy, urethane, silicone, silicone rubber, photopolymerizable compositions, vinyl-terminated monomers, gelatin resorcinol formaldehyde, gelatin resorcinol glutaraldehyde, anhydrides cross-linked with polyols, hyaluronic acid cross-linked with hydrazines, mixed monomer systems and co-polymers. For example, balloons, umbrellas and foam, as described herein, are particularly useful preformed polymerics. Deposited solutions are, for example, monomers or polymers in solution in which, after deposition of the solution on a surface, the solvent, such as a biocompatible solvent, is evaporated or dissipated leaving behind the monomer or polymer that was in solution. Viscous liquids, semi-solids, soft materials or solids may also be used, such as absorbable gelatin sponge (e.g., GelfoaMTM with liquid such as water or saline), hydrogels, latex, alginate compounds, waxes (absorbable or non-absorbable), petroleum-based compounds such as petrolatum, or various polymers in solvents, such as biocompatible solvents. Suitable sugars, alcohols, esters, acetates, starches, etc. could also be used for this purpose. Mechanical devices such as stents may be used to help anchor any one or more of the occlusive devices. For example, a lattice-work stent can provide a very strong anchor for an in situ-formed, e.g., polymerizable, occlusive device.

Various preformed foams may be used to occlude the lumen of a bronchial tube. Preferably, the foams are spongy and/or porous. Also a bronchial occlusion product may be provided comprising a compressible foam having interstices and an exterior; and a polymerizable material contained within or on at least one of the foam interstices and foam exterior. The foam may be shaped to allow said foam to be wedged in a bronchial tube. The foam may be impregnated with a polymerization initiator or accelerator, preferably compatible with various polymerizable materials.

Various medical balloons, such as used for balloon catheterization, constructed of, for example, silicone or latex, may also be used to occlude the lung. The occlusion balloon may be inflated before, or preferably after, it is placed in the desired location to occlude the lung. The balloon may be a variety of sizes when inflated, such as but not limited to balloons having diameters ranging in size from 0.5 to 50 mm, preferably from 1 to 40 mm, more preferably from 1.5 to 30 mm, even more preferably from 3 to 20 mm, even more preferably from 4 to 10 mm, and even more preferably from 5 to 7 mm, for example 4 mm, 5 mm, 6 mm, 7 mm, 8 mm or larger, to occlude different sized bronchial tubes. Due to the expandable property of such balloons, the balloons do not need to be sized specifically for a particular size bronchial tube. The balloon may be a variety of shapes, including but not limited to spherical and cylindrical, provided that the balloon, when inflated within a bronchial tube, occludes the bronchial tube.

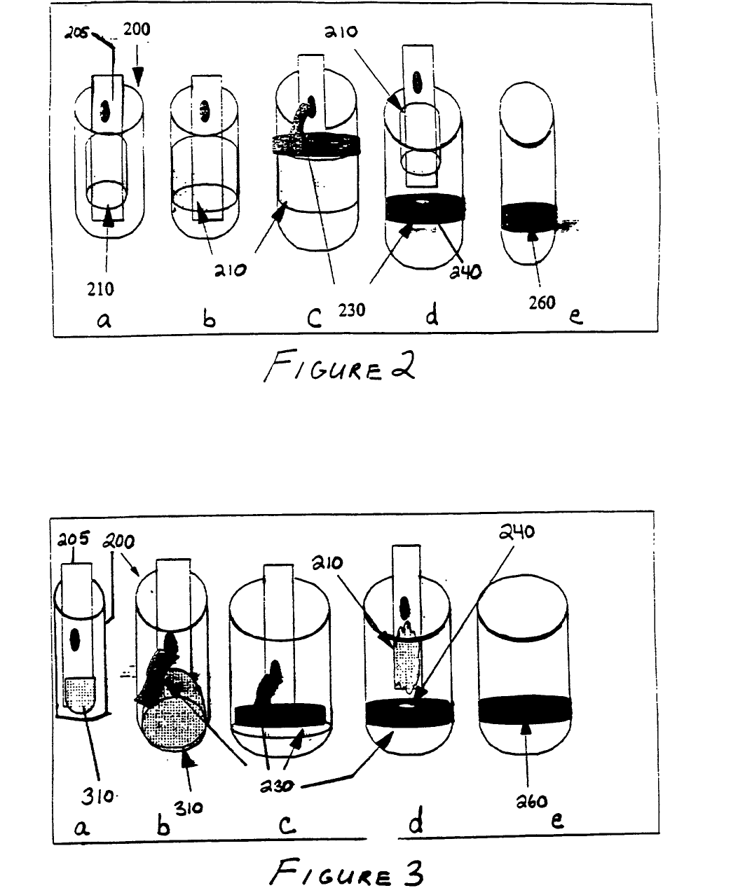

For example, FIG. 2 shows a cylindrical occlusion balloon 210 and FIG. 3 shows an inverted spherical occlusion balloon 310. Balloons 210 and 310 may be inserted into a bronchial tube 200 in any orientation, provided that balloons 210 and 310, when inflated within bronchial tube 200, occlude bronchial tube 200.

FIG. 2 shows exemplary steps of forming a bronchial occlusion. In step a, catheter 205 bearing occlusion balloon 210 is inserted into bronchial tube 200. Balloon 210 is then inflated in step b. In step c, polymerizable material 230 is introduced through catheter 205 onto the surface of inflated balloon 210. When polymerizable material 230 is self-supporting, balloon 210 is deflated and withdrawn through hole 240 as shown in step d. Additional polymerizable material or the like may be provided to fill hole 240, leaving a complete occlusion 260 as shown in step e. FIG. 3 shows a similar process, in which the same reference letters denote corresponding steps and the same reference numerals denote corresponding parts, using an inverted balloon 310. Alternatively, the polymerizable material 230 may be ejected from the catheter distal to the balloon 210. In this case, there is no need for hole 240, or it may merely be an indentation in the polymer plug that can be filled as the catheter is withdrawn.

Balloon 210 may be inflated and/or filled or coated with an adhesive, a polymerizable material which is allowed to polymerize, or any other suitable material, to provide additional support and permanency to the occlusion. It may also or alternatively be coated with a release agent, such as petroleum jelly. In embodiments, a polymerizable material 230 is formed on the inflated balloon as a polymer button plug, as shown in FIGS. 2 and 3, containing a hole 240 extending through polymerized material 230. Once the material has fully polymerized, the balloon can be deflated and retracted through the balloon retraction hole 240 present in the polymerized material. Once the balloon has been retracted, the remaining hole in the polymerized material can then be sealed off or filled in with the same or different polymerizable material to complete the occlusion 260.

In other embodiments of the invention, for example as shown in FIG. 4, in which the same reference letters denote corresponding steps and the same reference numbers denote corresponding parts, a small pliable umbrella 410 constructed of, for example, silicone or latex, may be delivered by catheter 205 to occlude a bronchial tube 200. The umbrella may be a variety of sizes when expanded, such as but not limited to umbrellas having diameters ranging in size from 0.5 to 50 mm, preferably from 1 to 40 mm, more preferably from 1.5 to 30 mm, even more preferably from 3 to 20 mm, even more preferably from 4 to 10 mm, and even more preferably from 5 to 7 mm, for example 4 mm, 5 mm, 6 mm, 7 mm, 8 mm or larger, to occlude different sized bronchial tubes. The occlusion umbrella 410 may be used in a variety of orientations, including but not limited to an extended/open orientation as shown in step b′ or a “wind-blown”/over-extended orientation as shown in step b″. Thus, a polymerizable material 230 could be added to the exterior of umbrella 410 or the interior of umbrella 410 could be filled with polymerizable material 230. When polymerizable material 230 polymerizes on umbrella 410, bronchial tube 200 is at least partially occluded (260). The umbrella may be left in place for additional support.

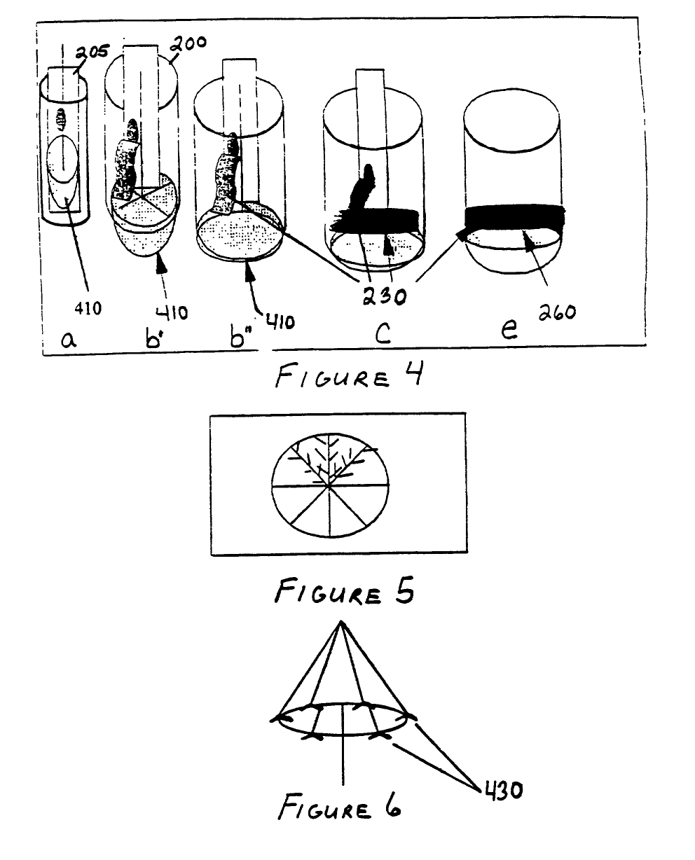

Umbrella 410 may additionally have claws (430) located at the distal end of each of the ribs or on the perimeter of umbrella 410 or may be coated with a polymerizable material, such as a cyanoacrylate adhesive, to secure umbrella 410 within bronchial tube 200. Umbrella 410 may have ribs of various materials, including but not limited to plastic, to provide stability and rigidity to the structure. Umbrella 410 may or may not have solid material spanning the ribs of umbrella 410 creating a canopy. In embodiments, a polymerizable material may be added to an umbrella skeleton to occlude a bronchial tube. For the purposes of this invention, an umbrella skeleton is an umbrella which lacks material fully spanning the region between the ribs. In embodiments, the umbrella could be constructed as an umbrella skeleton with additional protrusions from the ribs for added surface area creating a “snow-flake” design as partially shown in FIG. 5. A polymerizable material could then be applied to the ribs and protrusions of the “snow-flake” design and allowed to polymerize to occlude the bronchial tube.

In other embodiments of the invention, staples, clips, clamps and/or sutures, alone or in combination with the above bronchial occluders, may be used. For example, staples, clips, clamps and/or sutures may be used to provide a collapsing force on the exterior of a bronchial tube to occlude the bronchial tube and prevent air flow into a portion of lung. Generally, staples, clips, clamps and/or sutures are used on the exterior of a bronchial tube of the affected lung during open surgery or thoracotomy to apply an external force or pressure to collapse the bronchial tube. However, such staples, clips, clamps and sutures may also be used internally in a non-invasive or less-invasive endoscopic or laparoscopic procedure to occlude a region of affected lung tissue, for example with hooked needles to initiate the occlusion and then, for example, followed up with an adhesive. In preferred aspects of such embodiments, adhesives are also used to completely seal off air flow.

According to preferred embodiments of the present invention, a polymerizable material, such as a polymerizable monomer, is used as a bronchial occluder. A polymerizable material, such as a polymerizable monomer, that forms or can be made to form a polymer in situ may be used to occlude a lumen of a bronchial tube according to the present disclosure. Suitable polymerizable materials may be, for example, monomers and monomer systems, cyanoacrylate, acrylate, epoxy, urethane, silicone, silicone rubber, photopolymerizable compositions, vinyl-terminated monomers, gelatin resorcinol formaldehyde, gelatin resorcinol glutaraldehyde, anhydrides cross-linked with polyols, hyaluronic acid cross-linked with hydrazines, mixed monomer systems and co-polymers. Particularly suitable polymerizable materials, such as polymerizable monomers, and the polymerization products thereof expand under certain conditions, such as with heat, or with an added agent, such as a foaming agent.

According to embodiments of the present invention, a polymerizable adhesive is preferably used. Various adhesives, such as fibrin glue and preferably polymerizable 1,1-disubstituted ethylene adhesives such as monomeric cyanoacrylate adhesive, may be used in the present invention. An adhesive may be used alone or in conjunction with a solid device. For example, a small amount of wetted sterile surgical absorbable gelatin sponge or a small piece of sterile surgical foam may be introduced into a bronchial tube to at least partially fill the lumen and then the adhesive may be instilled into the bronchial tube under endoscopic guidance. Any of the above bronchial occluders may be coated with an adhesive, such as a 1,1-disubstituted ethylene monomer adhesive, or an adhesive may be placed on the interior or exterior surface of the bronchial tube to secure the bronchial occluder to the bronchial tube.

Monomer (including prepolymeric) compositions useful in this invention may include one or more polymerizable monomers.

Monomers that may be used in this invention include those that are readily polymerizable, e.g. anionically polymerizable or free radical polymerizable, or polymerizable by zwitterions or ion pairs to form polymers.

For example, polymerizable 1,1-disubstituted ethylene monomers, and adhesive compositions comprising such monomers, are disclosed in U.S. Pat. Nos. 6,010,714; 5,582,834; 5,575,997; 5,514,372; 5,514,371 and 5,328,687 to Leung et al. and 5,981,621 to Clark et al., the disclosures of which are hereby incorporated in their entirety by reference.

Useful 1,1-disubstituted ethylene monomers include, but are not limited to, monomers of the formula:

HRC═CXY (I)

wherein X and Y are each strong electron withdrawing groups, and R is H, —CH═CH₂ or, provided that X and Y are both cyano groups, a C₁-C₄ alkyl group.

Examples of monomers within the scope of formula (I) include α-cyanoacrylates, preferably alkyl-2-cyanoacrylates, vinylidene cyanides, C₁-C₄ alkyl homologues of vinylidene cyanides, dialkyl methylene malonates, acylacrylonitriles, vinyl sulfinates and vinyl sulfonates of the formula CH₂═CX′Y′ wherein X′ is —SO₂R′ or —SO₃R′ and Y′ is —CN, —COOR′, —COCH₃, —SO₂R′ or —SO₃R′, and R′ is H or hydrocarbyl.

Preferred monomers for use in this invention are alkyl α-cyanoacrylates. Such monomers are known in the art and have the formula CN

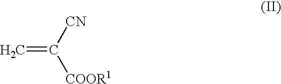

wherein R¹ is an alkyl or substituted alkyl group, a hydrocarbyl or substituted hydrocarbyl group; a group having the formula —R⁴—O—R⁵—O—R⁶, wherein R⁴ is a 1,2-alkylene group having 2-4 carbon atoms, R⁵ is an alkylene group having 2-4 carbon atoms, and R⁶ is an alkyl group having 1-6 carbon atoms; or a group having the formula

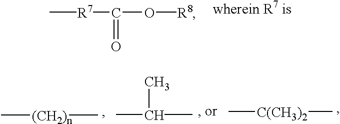

wherein n is 1-10, preferably 1-5 carbon atoms and R⁸ is an organic moiety.

Examples of suitable alkyl and substituted alkyl groups include straight chain or branched chain alkyl groups having 1-16 carbon atoms; and straight chain or branched chain C₁-C₁₆ alkyl groups substituted with a haloalkyl group, a halogen atom, a cyano group, or a haloalkyl group.

Examples of suitable hydrocarbyl and substituted hydrocarbyl groups include straight chain or branched chain alkyl groups having 1-16 carbon atoms; straight chain or branched chain C₁-C₁₆ alkyl groups substituted with an acyloxy group, a haloalkyl group, an alkoxy group, a halogen atom, a cyano group, or a haloalkyl group; straight chain or branched chain alkenyl groups having 2 to 16 carbon atoms; straight chain or branched chain alkynyl groups having 2 to 12 carbon atoms; cycloalkyl groups; aralkyl groups; alkylaryl groups; and aryl groups.

The organic moiety R⁸ may be substituted or unsubstituted and may be straight chain, branched or cyclic, saturated, unsaturated or aromatic. Examples of such organic moieties include C₁-C₈ alkyl moieties, C₂-C₈ alkenyl moieties, C₂-C₈ alkynyl moieties, C₃-C₁₂ cycloaliphatic moieties, aryl moieties such as phenyl and substituted phenyl and aralkyl moieties such as benzyl, methylbenzyl, and phenylethyl. Other organic moieties include substituted hydrocarbon moieties, such as halo (e.g., chloro-, fluoro- and bromo-substituted hydrocarbons) and oxy-substituted hydrocarbon (e.g., alkoxy substituted hydrocarbons) moieties. Preferred organic radicals are alkyl, alkenyl, and alkynyl moieties having from 1 to about 8 carbon atoms, and halo-substituted derivatives thereof. Particularly preferred are alkyl moieties of 4 to 6 carbon atoms.

In the cyanoacrylate monomer of formula (II), R¹ is preferably an alkyl group having 1-10 carbon atoms or a group having the formula —AOR⁹, wherein A is a divalent straight or branched chain alkylene or oxyalkylene moiety having 2-8 carbon atoms, and R⁹ is a straight or branched alkyl moiety having 1-8 carbon atoms.

Examples of groups represented by the formula —AOR⁹ include 1-methoxy-2-propyl, 2-butoxy ethyl, isopropoxy ethyl, 2-methoxy ethyl, and 2-ethoxy ethyl.

Exemplary α-cyanoacrylate monomers used in this invention are alkyl α-cyanoacrylates including octyl cyanoacrylate, such as 2-octyl cyanoacrylate; dodecyl cyanoacrylate; 2-ethylhexyl cyanoacrylate; butyl cyanoacrylate such as n-butyl or isobutyl cyanoacrylate; ethyl cyanoacrylate; and methyl cyanoacrylate. More preferred monomers are n-butyl and 2-octyl cyanoacrylate. Monomers utilized for medical purposes in the present invention should be very pure and contain few impurities (e.g., surgical grade).

The α-cyanoacrylates of formula (II) may be prepared according to methods known in the art. U.S. Pat. Nos. 2,721,858 and 3,254,111, each of which is hereby incorporated in its entirety by reference, disclose methods for preparing α-cyanoacrylates. For example, the α-cyanoacrylates may be prepared by reacting an alkyl cyanoacetate with formaldehyde in a non-aqueous organic solvent and in the presence of a basic catalyst, followed by pyrolysis of the anhydrous intermediate polymer in the presence of a polymerization inhibitor. The α-cyanoacrylate monomers prepared with low moisture content and essentially free of impurities are preferred for biomedical use.

A variety of polymerization, set or cure times can be produced by varying the type and/or amount polymerizable material, such as a polymerizable monomer, and/or by varying the type and/or concentration of various additives or initiators added to the polymerizable material. Polymerization, set or cure times may be on the order of 1 to 2 hours or shorter, such as 15-25 minutes or even 10 minutes. Preferably, the polymerization, set or cure times are 30 seconds to 15 minutes, and more preferably 1, 2, 3, 4, 5 or 6 minutes, such as 30-90 seconds, 120, 150 or 180 seconds.

Various monomers, particularly cyanoacrylate monomers, may be mixed with organic liquids or foaming agents, and preferably initiators, to form a composition that polymerizes and expands into, for example, a polycyanoacrylate foam. Suitable foaming agents include pentane, hexane, heptane, 1,1,2-trichlorotrifluoro-ethane, 1,1,1- trichlorotrifluoroethane, petroleum ether, diethyl ether, cyclopentane, cyclohexane, benzene, carbon tetrachloride, chloroform, methylcyclopentane, dimethylsulfide, 1,1-dichloroethane, 1,1,1-trichloroethane, perfluorohexane, perfluoroheptane, and 1-bromopropane. Examples of compositions that form polcyanoacrylate foams are disclosed in WO 92/09651, the entire disclosure of which is hereby incorporated in its entirety by reference.

A monomer, particularly a cyanoacrylate monomer, could be contained within an aerosol can and expelled via a pressurized gas to induce foaming. The expandable pressurized gas would cause the material to foam and expand. The aerosol can could be any conventional aerosol can or other dispensing apparatus, or a two-chambered spray foaming apparatus for delivering the unmixed elements. A surfactant could be further added to the mixture prior to dispensing to initiate polymerization and/or to carry additional compounds, drugs, or active agents to be incorporated into the polymer or delivered to the lung

The present invention also provides a method of achieving lung volume reduction comprising mixing a thickener with a biocompatible composition comprising at least one monomer that forms a medically acceptable polymer to form a mixture, and introducing said mixture into a lumen of a bronchial tube of a lung to prevent air flow to at least a region of the lung.

Various thickening agents may be added and may be selected from among thickeners, including, but not limited to, fimed silica, poly(2-ethylhexyl methacrylate), poly(2-ethylhexyl acrylate) and cellulose acetate butyrate. Suitable thickeners include, for example, polycyanoacrylates, polyoxalates, lactic-glycolic acid copolymers, polycaprolactone, lactic acid-caprolactone copolymers, poly(caprolactone+DL-lactide+glycolide), polyorthoesters, polyalkyl acrylates, copolymers of alkylacrylate and vinyl acetate, polyalkyl methacrylates, and copolymers of alkyl methacrylates and butadiene. Examples of alkyl methacrylates and acrylates include poly(butylmethacrylate) and poly(butylacrylate), also copolymers of various acrylate and methacrylate monomers, such as poly(butylmethacrylate-co-methylmethacrylate). Biodegradable polymer thickeners are preferred for some uses such as with absorbable adhesives. Preferably, the thickening agent is soluble in a monomer composition at room temperature (i.e., 20-25° C.) so that it may be added to the monomer composition without excessive heating of the monomer composition and remain uniformly combined in the composition.

Compositions useful in this invention may include at least one thixotropic agent. Suitable thixotropic agents are known to the skilled artisan and include, but are not limited to, fumed silica and silica gels such as those treated with a silyl isocyanate. In embodiments, biodegradable thixotropic agents, such as a cellulosic based material, may also be used. Examples of suitable thixotropic agents are disclosed in, for example, U.S. Pat. No. 4,720,513, the disclosure of which is hereby incorporated in its entirety.

Thickeners and/or thixotropic agents such as fumed silica with or without surface treatment can be added in a weight ratio of from about 1:5 to about 1:12 parts thickener to parts liquid in the formulation (e.g., plasticizer and monomer combined). The resultant material is gel-like and does not flow, or flows very little. For example, the material may be inverted in an open container without flowing from its container. Preferably, the weight ratio of such thickener to liquid in the formulation is from about 1:8 to 1:10. Most preferably, the weight ratio is about 1:8.5.

Various initiators may also be used in the present invention. Suitable initiators include, but are not limited to, detergent compositions; surfactants: e.g., nonionic surfactants such as polysorbate 20 (e.g., Tween 20™ from ICI Americas), polysorbate 80 (e.g., Tween 80±from ICI Americas) and poloxamers, cationic surfactants such as tetrabutylammonium bromide, butyrylcholine chloride, anionic surfactants such as sodium tetradecyl sulfate, and amphoteric or zwitterionic surfactants such as dodecyldimethyl(3-sulfopropyl)ammonium hydroxide, inner salt; amines, imines and amides, such as imidazole, tryptamine, urea, arginine and povidine; phosphines, phosphites and phosphonium salts, such as triphenylphosphine and triethyl phosphite; alcohols such as ethylene glycol, methyl gallate, ascorbic acid, tannins and tannic acid; inorganic bases and salts, such as sodium bisulfite, magnesium hydroxide, calcium sulfate and sodium silicate; sulfur compounds such as thiourea and polysulfides; polymeric cyclic ethers such as monensin, nonactin, crown ethers, calixarenes and polymeric epoxides; cyclic and acyclic carbonates, such as diethyl carbonate; phase transfer catalysts such as Aliquat 336; organometallics such as cobalt naphthenate and manganese acetylacetonate; and radical initiators and radicals, such as di-t-butyl peroxide and azobisisobutyronitrile. The polymerizable and/or cross-linkable material may also contain an initiator that is inactive until activated by a catalyst or accelerator.

To improve the cohesive strength of polymers and adhesives formed from compositions useful in this invention, difunctional monomeric cross-linking agents may be used with the monomer compositions. Such crosslinking agents are known. U.S. Pat. No. 3,940,362 to Overhults, which is hereby incorporated in its entirety by reference, discloses such cross-linking agents. Examples of suitable crosslinking agents include alkyl bis(2-cyanoacrylates), triallyl isocyanurates, alkylene diacrylates, alkylene dimethacrylates, trimethylol propane triacrylate, and alkyl bis(2-cyanoacrylates). A catalytic amount of an amine activated free radical initiator or rate modifier may be added to initiate polymerization or to modify the rate of polymerization of the cyanoacrylate monomer/crosslinking agent blend.

According to embodiments of this invention, a polymerizable monomer or adhesive initiator, for example butyrylcholine chloride, may be deposited onto a solid thickener, such as fumed silica, by pouring a solution of initiator over a specific amount of, for example, fumed silica. The solvent may then be removed, preferably by evaporation, leaving the initiator deposited onto the solid thickener. The level of initiator deposited on the solid thickener can be varied to make a more or less “concentrated” treated thickener. When mixed with a polymerizable monomer or adhesive such as cyanoacrylate, the treated solid thickener causes the polymerizable monomer or adhesive to begin polymerization. A variety of polymerization, set or cure times can be produced by varying the amount of treated solid thickener added and/or by varying the concentration of the initiator in the initial solution used to treat the solid thickener. Polymerization, set or cure times may be on the order of 1 to 2 hours or shorter, such as 15-25 minutes or even 10 minutes. Preferably, the polymerization, set or cure times are 30 seconds to 15 minutes, and more preferably 1, 2, 3, 4, 5 or 6 minutes, such as 30-90 seconds, 120, 150 or 180 seconds.

In embodiments, fumed silica surface treated with, for example, dimethyl silicone to produce a surface containing polydimethyl siloxane polymer may be mixed with a polymerizable monomer of the invention prior to initiating. Such premixing may increase the dispersion of the various additives in the monomer and may assist in achieving uniform polymerization. The fiuned silica may be in amounts up to 20%, such as up to 15%, such as up to 12%, for example approximately 10.5% by weight of the total composition.

Compositions useful in this invention may optionally also include at least one plasticizing agent that imparts flexibility to the polymer formed from the monomer. The plasticizing agent preferably contains little or no moisture and should not significantly affect the stability or polymerization of the monomer. Some thickeners, such as poly-2-ethylhexylcyanoacrylate, can also impart flexibility to the polymer.

The addition of plasticizing agents in amounts ranging from about 0.5 wt. % to about 60 wt. %, or from about 1 wt. % to about 60 wt. %, or from about 3 wt. % to about 50 wt. % or from about 5 wt. % to about 50 wt. % based on the weight of the monomer and plasticizer provides increased elongation and toughness of the polymerized monomer over polymerized monomers not having plasticizing agents.

Examples of suitable plasticizers include acetyl tributyl citrate, dimethyl sebacate, triethyl phosphate, tri(2-ethylhexyl)phosphate, tri(p-cresyl) phosphate, glyceryl triacetate, glyceryl tributyrate, diethyl sebacate, dioctyl adipate, isopropyl myristate, butyl stearate, lauric acid, trioctyl trimellitate, dioctyl glutarate, polydimethylsiloxane, and mixtures thereof. Preferred plasticizers are tributyl citrate and acetyl tributyl citrate. Suitable plasticizers include polymeric plasticizers, such as polyethylene glycol (PEG) esters and capped PEG esters or ethers, polyester glutarates and polyester adipates.

A preservative may be included in the composition to inhibit the growth of microorganisms including those that may be introduced into the composition during the surgery or procedure. Preservatives useful in compositions useful in this invention may be selected from among known anti-microbial agents. In embodiments, the preservative may be selected from among preservatives, including, but not limited to, parabens and cresols. For example, suitable parabens include, but are not limited to, alkyl parabens and salts thereof, such as methylparaben, methylparaben sodium, ethylparaben, propylparaben, propylparaben sodium, butylparaben, and the like. Suitable cresols include, but are not limited to, cresol, chlorocresol, and the like. The preservative may also be selected from other known agents including, but not limited to, hydroquinone, pyrocatechol, resorcinol, 4-n-hexyl resoreinol, captan (i.e., 3a,4,7,7a-tetrahydro-2-((trichloromethyl)thio)-1H-isoindole-1,3 (2H)-dione), benzalkonium chloride, benzalkonium chloride solution, benzethonium chloride, benzoic acid, benzyl alcohol, cetylpyridinium chloride, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, phenylmercuric compounds such as phenylmercuric borate, phenylmercurie nitrate and phenylmercuric acetate, formaldehyde, and formaldehyde generators such as the preservatives Germall II® and Germall 115™ (imidazolidinyl urea, available from Sutton Laboratories, Charthan, New Jersey). Other suitable preservatives are disclosed in U.S. patent application Ser. No. 09/430,180, filed Oct. 29, 1999, the entire disclosure of which is hereby incorporated by reference. In embodiments, mixtures of two or more preservatives may also be used.

Monomer compositions useful in the invention may be sterilized. The sterilization may be accomplished by techniques known to the skilled artisan, and is preferably accomplished by methods such as, but not limited to, chemical, physical, and/or irradiation methods. Examples of physical methods include, but are not limited to, sterile fill, filtration, sterilization by heat (dry or moist) and retort canning. Examples of irradiation methods include, but are not limited to, gamma irradiation, electron beam irradiation, and microwave irradiation. Preferred methods are dry and moist heat sterilization and electron beam irradiation. The sterilized composition should show low levels of toxicity to living tissue during its useable life.

Any of the bronchial occluders of the present invention, such as polymerizable materials, balloons, umbrellas etc., may be radiopaque or may contain or be coated with radiopaque additives to assist in non-intrusive (e.g., X-ray) visualization and monitoring of the occlusion. For example, monomer compositions useful in the invention may include radiopaque additives. A polymer formed from a composition containing radiopaque additives would be visible by x-ray visualization. The size or orientation of the polymer or other bronchial occluder could be visualized by an x-ray to determine whether the polymer had formed properly and/or whether the polymer or other bronchial occluder had shifted or moved. Examples of suitable radiopaque additives may be tantalum metal or other metals, barium compounds such as barium sulfate, organic iodo acids, particularly iodo carboxylic acids, triiodophenol, iodoform and tetraiodoethylene. In embodiments, iodine may be present in an amount of about 2-15 mole percent, preferably 7-10 mole percent of the monomer composition.

Monomer compositions useful in the invention may also include a heat dissipating agent. Heat dissipating agents include liquids or solids that may be soluble or insoluble in the monomer. The liquids may be volatile and may evaporate during polymerization, thereby releasing heat from the composition. Suitable heat dissipating agents may be found, for example, in U.S. Pat. No. 6,010,714 to Leung et al., the entire disclosure of which is incorporated herein.

Compositions useful in this invention may also optionally include stabilizing agents, preferably both at least one anionic vapor phase stabilizer and at least one anionic liquid phase stabilizer. These stabilizing agents inhibit premature polymerization. Such stabilizing agents may also include mixtures of anionic stabilizing agents and radical stabilizing agents. Any mixture of stabilizers is included as long as the mixture does not inhibit the desired polymerization of the monomer. Examples of stabilizing agents, and mixtures of stabilizing agents, are found in U.S. patent application Ser. No. 09/099,457 filed Jun. 18, 1998, the entire disclosure of which is hereby incorporated by reference.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties.

Compositions useful in this invention may further contain fibrous reinforcement and colorants such as dyes, pigments, and pigment dyes. Examples of suitable fibrous reinforcement include silk fibers, nylon fibers, PGA microfibrils, collagen microfibrils, cellulosic microfibrils, and olefinic microfibrils. Examples of suitable colorants include 1-hydroxy-4-[4-methylphenyl-amino]-9,10 anthracenedione (D+C violet No. 2); disodium salt of 6-hydroxy-5-[(4-sulfophenyl)axo] 2-naphthalene-sulfonic acid (FD+C Yellow No. 6); 9-(o-carboxyphenoyl)-6-hydroxy-2,4,5,7-tetraiodo-3H-xanthen-3-one, disodium salt, monohydrate (FD+C Red No.3); 2-(1,3-dihydro-3-oxo-5-sulfo-2H-indol-2-ylidene)-2,3-dihydro-3-oxo-1H-indole-5-sulfonic acid disodium salt (FD+C Blue No. 2); and [phthalocyaninato (2-)] copper.

Medical compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites; ammonium sulfite salts; amines; amides; imides; nitrites; carbamates; alcohols; mercaptans; proteins; mixtures of amines, amides, and proteins; active methylene compounds such as cyclic ketones and compounds having a b-dicarbonyl group; and heterocyclic ring compounds free of a carbonyl group and containing an NH group, with the ring made up of nitrogen or carbon atoms, the ring being unsaturated or, when fused to a phenyl group, being unsaturated or saturated, and the NH group being bonded to a carbon or a nitrogen atom, which atom is directly bonded by a double bond to another carbon or nitrogen atom.

Other examples of formaldehyde level reducing compounds and compositions are exemplified by U.S. Pat. Nos. 6,010,714; 5,624,669; 5,582,834; 5,575,997, the entire disclosures of which are hereby incorporated by reference.

Other compositions useful in the present invention are exemplified by U.S. Pat. Nos. 5,624,669; 5,582,834; 5,575,997; 5,514,371; 5,514,372; and 5,259,835; and U.S. patent application Ser. No. 08/714,288, the disclosures of all of which are hereby incorporated in their entirety by reference.

Suitable methods and applicators for applying such compositions to substrates, and particularly in medical applications, are described in, for example, U.S. Pat. Nos. 5,928,611; 5,582,834; 5,575,997; and 5,624,669, all to Leung et al. and U.S. patent application Ser. No. 09/450,686 filed Nov. 30, 1999, the disclosures of which are hereby incorporated in their entirety by reference.

Methods of the present invention utilizing polymerizable monomers, and preferably adhesive compositions, may be carried out in single or multiple applications. The monomers or adhesives may be applied in a first layer or plug, and after the first layer or plug is allowed to fully or partially polymerize, one or more subsequent layer or plug may be added on, adjacent to or spaced from a prior layer or plug. In some instances, a monomer or adhesive may be applied to the lumen of a bronchial tube, but the plug formed may not possess sufficient strength or adhesion to the bronchial wall to remain in place over an extended period of time. Therefore, a second or further application of the monomer or adhesive may serve to strengthen and thicken the occlusion. Such a process may be repeated numerous times, depending on the size of the lumen of the bronchial tube and the amount of polymerizable monomer or adhesive applied in each application. An initial application of the monomer or adhesive may also be applied such that an incomplete occlusion is formed on the first application. Therefore, additional applications of the monomer or adhesive to the monomer or adhesive applied in the first application may result in a complete occlusion of the lumen of the bronchial tube. Placement of a plurality of spaced plugs helps avoid leakage in the event that there is movement of or leakage around a single plug.

Complete occlusion can also be promoted by administration of an anti-secretory agent that reduces or prevents secretion of mucous in the lung or the portion of the lung being treated with the occluder. The anti-secretory agent can be administered prior to or even simultaneously with, on or in the occluder. Non-limiting examples of such anti-secretory agents include anticholinergic agents, atropine and atropinic agents, for example Robinul™ (glycopyrrolate).

Certain pre-treatments of the lung associated with occlusion according to the invention can also be advantageous. For example, lavage of the lung or affected portion of the lung with bioactive agents as described above can help by treating pre-existing conditions or by avoiding infection or the like associated with the occlusion procedure. Evacuation of mucous in the lung before such washing and/or before occlusion may also facilitate and improve the effectiveness of treatment.

Combination of occlusion with other medical treatments may also be advantageous. For example, where cancerous tissue such as a tumor is present, chemical or radioactive agents may be placed at such tissue in conjunction with the placement of one or more occlusive devices.

Preferably, an apparatus that allows for mixing various components prior to delivery into a bronchial tube may be used. Various apparatus may be used such as those disclosed in U.S. Pat. No. 5,928,611 to Leung, the entire disclosure of which is hereby incorporated by reference.

The present invention provides an apparatus for achieving lung volume reduction comprising a means for mixing at least one component with a biocompatible composition comprising at least one monomer that forms a medically acceptable polymer to form a mixture, and a means for introducing the mixture into a lumen of a bronchial tube of a lung to prevent air flow to at least a region of the lung.

The apparatus for mixing components comprises at least a first and second syringe removably attached to a mixing valve having at least a coupling point to connect each of said first and second syringe to said mixing valve; and at least a first and second plunger movable within each syringe, wherein the components are moved back and forth between the syringes by alternately depressing the plungers to mix the components prior to extruding the mixed components. For the purposes of this invention, the term “mixing valve” means a mixing apparatus or connector that allows for components to be mixed with each other and allows the components to move into and out of the mixing valve. Examples of mixing valves are three-way stopcocks.

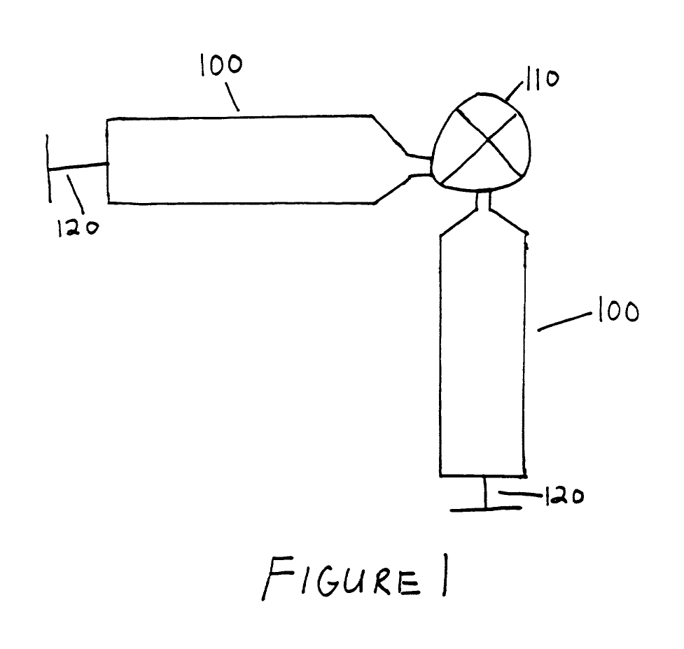

According to one aspect of this invention, a lung occlusion delivery system is provided. In the mixing apparatus of FIG. 1, two syringes 100 are used; preferably with different components in each syringe. For the purposes of the present invention, the term “syringe” means any instrument or device capable of holding at least one component and capable of injecting components out of and/or drawing components into the syringe. For example, one syringe may contain a liquid component, such as a polymerizable monomer or a cyanoacrylate adhesive, and the other may contain a solid, preferably powder, component, such as fumed silica, tantalum, and/or an initiator. Each syringe may contain a single component, or contain a mixture of components. In many cases, it may be beneficial to keep the components separate until polymerization, curing or reaction is desired. A particular benefit of the mixing apparatus is that incompatible materials may be introduced at the time of use, eliminating concerns about shelf-life or premature polymerization of the components.

Syringes 100 are preferably removably coupled to a mixing valve 110, which is preferably a three-way stopcock or other suitable means for mixing the components held in syringes 100. Preferably, syringes 100 have threaded dispensing ends to couple to threaded coupling points on mixing valve 110. This coupling provides additional stability to the apparatus during mixing. The contents of syringes 100 are mixed back and forth within syringes 100, as well as within mixing valve 110, by pressing on alternate plungers 120 of each syringe 100 to achieve the desired level of mixing, homogeneity, reactivity, or viscosity. Mixing valve 110 allows the components to move back and forth between syringes 100 for mixing. The mixture can then be pushed into a single syringe 100 for dispensing. Alternatively, mixing valve 110 may contain an opening for extruding the mixed contents. A syringe 100 or mixing valve 110 containing the mixture may be affixed to the end of an appropriate endoscopic catheter, needle, or similar device, for delivery of the mixture to the lumen of a bronchial tube. Other mixing devices can also be used.

Also, the mixing system may advantageously produce air bubbles or microbubbles in the mixture during the mixing process by vigorous mixing or the intentional introduction of air or other gas into the mixture. For example, there may be air space in at least one syringe that may be introduced into the mixture to produce air bubbles or microbubbles. Alternatively, the mixture or polymerizable material may be premixed with a gas such as air, oxygen, etc., to create bubbles in the mixture. If the liquid is polymerizable, as the material polymerizes and heats, the bubbles will expand within the mixture and expand the mixture mass, which is particularly beneficial to occlude a bronchial tube. Use of vacuum can also help create such bubbles.

### EXAMPLES

The present invention will be further understood by reference to the following non-limiting examples.

### Example 1

2.5 g fumed silica is covered with 40 ml of initiator solution containing butyrylcholine chloride in methanol. The solvent is allowed to evaporate leaving a solid material of fumed silica with butyrylcholine chloride deposited on the fumed silica. This material may be ground up into a powder and used as treated thickener to initiate polymerizable monomers.

### Example 2

2 ml of 2-octyl cyanoacrylate monomer/ATBC (100 parts to 6 parts) is added to a 20 ml glass scintillation vial. Treated thickener from Example 1 is added in consecutive runs. The amount of initiator varies in consecutive runs. Results are shown in the table below.

| | | | Concentration of | | | |
|---|---|---|---|---|---|---|
| | Amount of | | Initiator Solution for | | Approximate | |
| Run | Treated Thickener | | Treatment in Example 1 | | Gel Time | |
| 1 | 0.222 | g | 99.8 | ppm | <2 | hours |
| 2 | 0.222 | g | 497 | ppm | 3 | minutes |
| 3 | 0.222 | g | 1573 | ppm | 90 | seconds |
| 4 | 0.222 | g | 9746 | ppm | 40 | seconds |
| 5 | 0.15 | g and | 99.8 | ppm and | 15-25 | minutes |
| | 0.15 | g | 497 | ppm | | |
| 6 | 0.225 | g and | 497 | ppm and | 10 | minutes |
| | 0.075 | g | 99.8 | ppm | | |

### Example 3

To create a gel-like material, non-initiated fumed silica (as supplied off the shelf) is added to a 2-octyl cyanoacrylate/ATBC mixture. The ratio of fumed silica to 2-octyl cyanoacrylate/ATBC is 1:8.5. The ratio of 2-octyl cyanoacrylate to ATBC is 100 parts to 6 parts. Approximately 2.5 cc of this gel is transferred to a 3 cc syringe. To a second 3 cc syringe 0.0360 g of 9746 ppm treated fumed silica and 0.19 g tantalum powder are added. Prior to use the two syringes are coupled with a three way stopcock as shown in FIG. 1. The materials are mixed back and forth for approximately 30 seconds and then the mixture is deposited in the lumen of a bronchial tube of a goat and allowed to polymerize. The monomer polymerizes in the goat in approximately 40 seconds after placement. The lung is observed using x-ray visualization 3 months after application of the polymerizable monomer. The lung displays atelectasis in the blocked region of the lung.

### Example 4

To occlude a 5 mm wide region of a bronchial tube, a latex balloon, inflatable to a 6 mm diameter, is inserted into a bronchial tube. After the balloon is positioned in the desired location within the bronchial tube, the balloon is inflated until it occludes the bronchial tube. To the exterior uppermost exposed portion of the inflated balloon, a 2-octyl cyanoacrylate composition is added to cover the exposed region of the balloon and allowed to polymerize. The balloon is then deflated and withdrawn, and additional 2-octyl cyanoacrylate is then used to fill the withdrawal hole and allowed to polymerize to complete the occlusion.

### Example 5

To occlude a 5 mm wide region of a bronchial tube, an occlusion umbrella, expandable to a 6 mm diameter, is inserted into a bronchial tube. After the umbrella is positioned in the desired location within the bronchial tube, the umbrella is opened until it is secured within the bronchial tube. To the exterior uppermost exposed portion of the expanded umbrella, a 2-octyl cyanoacrylate composition is added to cover the exposed region of the umbrella and allowed to polymerize to complete the occlusion.

While the invention has been described with reference to preferred embodiments, the invention is not limited to the specific examples given, and other embodiments and modifications can be made by those skilled in the art without departing from the spirit and scope of the invention.

What is claimed is:

1. A method of achieving lung volume reduction, comprising:
occluding a lumen of a bronchial tube of a lung to substantially reduce or prevent air flow to at least a region of the lung without surgically removing said region of the lung.

2. The method of claim 1, wherein said occluding leads to deflation and atelactasis of said region of the lung.

3. The method of claim 1, wherein the occluding step includes introducing at least one bronchial occluder into said lumen to occlude said lumen.

4. The method of claim 3, wherein said bronchial occluder is introduced into said lumen at a bifurcation or branching of the lung.

5. The method of claim 1, comprising occluding one or more said lumen at spaced apart locations.

6. The method of claim 3, wherein said at least one bronchial occluder comprises a biocompatible composition comprising at least one monomer that forms a medically acceptable polymer.

7. The method of claim 6, wherein said at least one monomer is a 1,1-disubstituted ethylene monomer.

8. The method of claim 6, wherein said composition comprises at least one member selected from the group consisting of cyanoacrylate, acrylate, epoxy, urethane, silicone, silicone rubber, photopolymerizable compositions, vinyl-terminated monomers, gelatin resorcinol formaldehyde, gelatin resorcinol glutaraldehyde, anhydrides cross-linked with polyols, hyaluronic acid cross-linked with hydrazines, mixed monomer systems and co-polymers.

9. The method of claim 6, wherein said at least one monomer is an α-cyanoacrylate monomer.

10. The method of claim 6, wherein said at least one monomer comprises at least one member selected from the group consisting of ethyl cyanoacrylate, butyl cyanoacrylate, and 2-octyl cyanoacrylate.

11. The method of claim 6, wherein said at least one monomer is 2-octyl cyanoacrylate.

12. The method of claim 6, wherein said at least one monomer polymerizes in 30 seconds to 15 minutes.

13. The method of claim 6, wherein said at least one monomer polymerizes in 1 to 6 minutes.

14. The method of claim 3, wherein said bronchial occluder is radiopaque or contains a radiopaque additive.

15. The method of claim 6, wherein said biocompatible composition further comprises a thickener.

16. The method of claim 15, wherein an initiator is deposited on said thickener.

17. The method of claim 16, wherein said initiator is butyrylcholine chloride.

18. The method of claim 16, wherein said thickener is fumed silica.

19. The method of claim 3, wherein said at least one bronchial occluder has a Sterility Assurance Level (SAL) of from 10⁻³ to 10⁻⁶.

20. The method of claim 3, wherein said at least one bronchial occluder comprises at least one member selected from the group consisting of polymerizable compositions, preformed solid polymerics, deposited solutions, viscous liquids, semi-solids and solids.

21. The method of claim 20, wherein said at least one bronchial occluder further comprises at least one bioactive agent.

22. The method of claim 21, wherein said at least one bioactive agent is selected from the group consisting of antibiotics, antimicrobials, antiseptics, bacteriocins, bacteriostats, disinfectants, steroids, anesthetics, fungicides, anti-inflammatory agents, antibacterial agents, antiviral agents, antitumor agents and growth promoting substances.

23. The method of claim 1, further comprising administering an anti-secretory agent to reduce secretions in said lung that might interfere with said occluding.

24. The method of claim 1, further comprising washing said lung with a bioactive agent before said occluding.

25. The method of claim 3, wherein said at least one bronchial occluder comprises a viscous liquid.

26. The method of claim 3, wherein said at least one bronchial occluder comprises a deposited solution.

27. The method of claim 3, wherein said at least one bronchial occluder comprises a preformed polymeric device.

28. The method of claim 3, wherein said at least one bronchial occluder comprises an inflatable balloon, umbrella or iris diaphragm.

29. The method of claim 28, comprising at least partially filling or coating said balloon or said umbrella with a biocompatible composition comprising at least one monomer that forms a medically acceptable polymer.

30. The method of claim 29, wherein said at least one monomer is a 1,1-disubstituted ethylene monomer.

31. The method of claim 29, wherein said at least one monomer is an α-cyanoacrylate monomer.

32. The method of claim 28, comprising opening said umbrella within said lumen to occlude said lumen.

33. The method of claim 32, wherein said umbrella is opened in a wind-blown inverted direction.

34. The method of claim 32, comprising at least partially filling or coating said umbrella with a biocompatible composition comprising at least one monomer that forms a medically acceptable polymer.

35. The method of claim 32, wherein said umbrella further comprises a canopy covering a plurality of ribs extending radially from a center shaft of said umbrella.

36. The method of claim 35, wherein said plurality of ribs further comprises a plurality of protrusions.

37. The method of claim 35, wherein a biocompatible composition comprising at least one monomer that forms a medically acceptable polymer is applied to said canopy to occlude said lumen.

38. The method of claim 36, wherein a biocompatible composition comprising at least one monomer that forms a medically acceptable polymer is applied to said plurality of ribs and said plurality of protrusions to occlude said lumen.

39. The method of claim 29, further comprising:
inflating said balloon within said lumen prior to applying said biocompatible composition;
allowing said biocompatible composition to polymerize on said inflated balloon with a hole in the center of the polymerized biocompatible composition;
deflating said balloon and withdrawing the balloon through the hole in the center of the polymerized biocompatible composition and withdrawing the balloon from said lumen; and
filling said hole in the center of the polymerized biocompatible composition with a second polymerizable biocompatible composition.

40. The method of claim 39, wherein said second polymerizable biocompatible composition is the same composition as the polymerized biocompatible composition applied to the balloon.

41. The method of claim 3, comprising leaving said at least one bronchial occluder permanently resident in said lumen.

42. The method of claim 3, wherein said at least one bronchial occluder is semi-permanently resident in said lumen.

43. The method of claim 1, wherein said lung is in a patient suffering from a pulmonary disorder or disease.

44. The method of claim 43, wherein said pulmonary disorder or disease is emphysema.

45. The method of claim 43, wherein said pulmonary disorder or disease is a fistula.

46. The method of claim 1, wherein the occluding step comprises collapsing a bronchial tube of a lung with an external force applied to the bronchial tube to occlude the bronchial tube.

47. The method of claim 46, wherein said external force is applied by at least one step selected from the group consisting of suturing the bronchial tube, and clamping the bronchial tube.

48. The method of claim 47, wherein the occluding step further comprises applying a biocompatible polymer to the bronchial tube.

49. The method of claim 48, wherein said biocompatible polymer is a poly-α-cyanoacrylate.

50. A method of achieving lung volume reduction, comprising:
mixing a thickener or filler with a biocompatible composition comprising at least one monomer that forms a medically acceptable polymer to form a mixture; and
introducing said mixture into a lumen of a bronchial tube of a lung to occlude said lumen and thereby prevent air flow to at least a region of the lung.

51. The method of claim 50, wherein said thickener is fumed silica.

52. The method of claim 50, wherein an initiator is added to said thickener prior to mixing said thickener with said composition.

53. The method of claim 52, wherein said initiator is added to said thickener by adding a solvent containing the initiator to the thickener and then evaporating the solvent.

54. The method of claim 53, wherein said thickener is fumed silica.

55. The method of claim 51, wherein said at least one monomer is a 1,1-di substituted ethylene monomer.

56. The method of claim 51, wherein said at least one monomer is an α-cyanoacrylate monomer.

57. The method of claim 51, wherein said at least one monomer comprises at least one member selected from the group consisting of ethyl cyanoacrylate, butyl cyanoacrylate, and 2-octyl cyanoacrylate.

58. The method of claim 51, wherein said at least one monomer is 2-octyl cyanoacrylate.

59. The method of claim 50, wherein said at least one monomer polymerizes in 30 seconds to 15 minutes.

60. The method of claim 50, wherein said at least one monomer polymerizes in 1 to 6 minutes.

61. The method of claim 50, wherein said biocompatible composition further comprises a radiopaque additive.

62. An apparatus for achieving lung volume reduction, comprising:
means for mixing at least one component with a biocompatible composition comprising at least one monomer that forms a medically acceptable polymer to form a mixture; and
means for introducing said mixture into a lumen of a bronchial tube of a lung to occlude said lumen and thereby substantially reduce or prevent air flow to at least a region of the lung.

63. An apparatus for mixing components, comprising:
first and second syringes removably attached to a mixing valve having at least one coupling point to connect each of said first and second syringes to said mixing valve; and
first and second plungers respectively movable within said first and second syringes, wherein the components are moved back and forth between the syringes by alternately depressing the plungers to mix the components prior to extruding the mixed components.

64. The apparatus of claim 63, wherein each of said first and second syringes has a threaded dispensing end and said mixing valve has first and second complementary threaded coupling points to receive said dispensing ends.

65. The apparatus of claim 63, wherein said mixing valve has an opening for extruding the mixed components.

66. A method of achieving lung volume reduction, comprising:
mixing a first component and a second component in the apparatus of claim 63, wherein at least one of said first and second components is polymerizable, to form a mixture;
introducing said mixture into a lumen of a bronchial tube; and
allowing said polymerizable component to polymerize and occlude the bronchial tube.

67. The method of claim 66, further comprising:
prior to mixing said first and second components, drawing at least one of said first and second components into a syringe; and
drawing a quantity of air into the same syringe.

68. The method of claim 66, wherein at least one of said first and second components is a biocompatible composition comprising at least one monomer that forms a medically acceptable polymer.

69. The method of claim 68, wherein said at least one monomer is a 1,1-disubstituted ethylene monomer.

70. The method of claim 68, wherein said at least one monomer is an α-cyanoacrylate monomer.

71. The method of claim 68, wherein said at least one monomer comprises at least one member selected from the group consisting of ethyl cyanoacrylate, butyl cyanoacrylate, and 2-octyl cyanoacrylate.

72. The method of claim 68, wherein said at least one monomer is 2-octyl cyanoacrylate.

73. The method of claim 68, wherein said at least one monomer polymerizes in 30 seconds to 15 minutes.

74. The method of claim 68, wherein said at least one monomer polymerizes in 1 to 6 minutes.

75. The method of claim 68, wherein said biocompatible composition further comprises a radiopaque additive in an amount effective to assist in non-intrusive visualization of said composition.

76. The method of claim 68, wherein said second component comprises a polymerization initiator or accelerator for said monomer.

77. The method of claim 68, wherein said at least one monomer is a monomer premixed with air.

78. A stable composition, comprising:
a thickener compatible with a polymerizable monomer; and
an initiator or accelerator for promoting polymerization of said polymerizable monomer, said composition being substantially free of said monomer.

79. The composition of claim 78, wherein said initiator or accelerator is at least partially coated on said thickener.

80. The composition of claim 78, wherein said polymerizable monomer is a 1,1-disubstituted ethylene monomer.

81. The composition of claim 79, wherein said polymerizable monomer is an α-cyanoacrylate monomer.

82. The composition of claim 78, wherein said thickener is fumed silica.

83. The composition of claim 78, wherein said initiator or accelerator is butyrylcholine chloride.

84. A kit comprising a saleable package comprising:
a first container that contains at least one polymerizable monomer; and
a second container that contains a composition comprising a thickener compatible with said polymerizable monomer and an initiator or accelerator for promoting polymerization of said polymerizable monomer, said composition contained in said second container being substantially free of said monomer.

85. The kit of claim 84, wherein said initiator or accelerator is at least partially coated on said thickener.

86. The kit of claim 84, wherein said polymerizable monomer is a 1,1-disubstituted ethylene monomer.

87. The kit of claim 84, wherein said polymerizable monomer is an α-cyanoacrylate monomer.

88. The kit of claim 84, wherein said polymerizable monomer comprises at least one member selected from the group consisting of ethyl cyanoacrylate, butyl cyanoacrylate, and 2-octyl cyanoacrylate.

89. The kit of claim 84, wherein said thickener is fumed silica.

90. The kit of claim 84, wherein said initiator or accelerator is butyrylcholine chloride.

91. The kit of claim 84, further comprising:
a bronchial occluder selected from the group consisting of foam, sponge, balloons, umbrellas and ball bearings.

92. The kit of claim 84, wherein said kit has a Sterility Assurance Level (SAL) of 10⁻³ to 10⁻⁶.

93. A bronchial occlusion product, comprising:
a compressible foam having interstices and an exterior; and
a polymerizable monomer contained within or on at least one of said foam interstices and said foam exterior.

94. The bronchial occlusion product of claim 93, wherein said foam is shaped to allow said foam to be wedged in a bronchial tube.

95. The bronchial occlusion product of claim 93, wherein said polymerizable monomer is a 1,1-disubstituted ethylene monomer.

96. The bronchial occlusion product of claim 93, wherein said polymerizable monomer is an α-cyanoacrylate monomer.

97. The bronchial occlusion product of claim 93, wherein said polymerizable monomer comprises at least one member selected from the group consisting of ethyl cyanoacrylate, butyl cyanoacrylate, and 2-octyl cyanoacrylate.

98. A kit comprising a saleable package comprising:
a first container that contains at least one polymerizable monomer; and
a second container that contains a preformed physical bronchial occluder.

99. The kit of claim 98, wherein said preformed physical bronchial occluder is at least one member selected from the group consisting of foam, balloons, umbrellas and ball bearings.

100. The kit of claim 98, wherein said polymerizable monomer is a 1,1-disubstituted ethylene monomer.

101. The kit of claim 98, wherein said polymerizable monomer is an α-cyanoacrylate monomer.

102. The kit of claim 98, wherein said polymerizable monomer comprises at least one member selected from the group consisting of ethyl cyanoacrylate, butyl cyanoacrylate, and 2-octyl cyanoacrylate.

103. The kit of claim 98, further comprising an intrabronchial applicator for said monomer.

104. The kit of claim 98, wherein said preformed physical bronchial occluder is foam, wherein said foam is impregnated with a polymerization initiator or accelerator compatible with said polymerizable monomer.

105. A bronchial occluder, comprising:
an expandable umbrella having a center shaft and a plurality of ribs extending outward from said center shaft.

106. The bronchial occluder of claim 105, wherein said plurality of ribs each have a distal end and wherein said distal end of each rib further comprises a claw.

107. The bronchial occluder of claim 105, wherein said expandable umbrella further comprises a canopy covering said plurality of ribs.

108. The bronchial occluder of claim 107, wherein said canopy has an outer perimeter and wherein said outer perimeter further comprises claws.

109. The bronchial occluder of claim 107, wherein said plurality of ribs further comprises a plurality of protrusions.

110. The bronchial occluder of claim 105, wherein said expandable umbrella has a diameter of from 5 mm to 7 mm when expanded.

111. The bronchial occluder of claim 105, wherein said expandable umbrella has a Sterility Assurance Level (SAL) of from 10⁻³ to 10⁻⁶.

112. A bronchial occlusion apparatus, comprising:
a dispensing container containing a polymerizable monomer and a pressurized gas compatible with and stable in combination with said polymerizable monomer.

113. The bronchial occlusion apparatus of claim 112, wherein said polymerizable monomer is contained within a first chamber within said dispensing container; and
at least one component selected from the group consisting of a thickener, an initiator, a plasticizer, a radiopaque additive, a colorant, a preservative, a heat dissipating agent, a surfactant, and a formaldehyde scavenger is contained within a second chamber within said dispensing container.

114. The bronchial occlusion apparatus of claim 112, wherein said dispensing container is an aerosol can.

115. The bronchial occlusion apparatus of claim 113, wherein said first chamber further comprises a foaming agent.

116. The bronchial occlusion apparatus of claim 115, wherein said foaming agent is at least one member selected from the group consisting of pentane, hexane, heptane, 1,1,2-trichlorotrifluoroethane, 1,1,1- trichlorotrifluoroethane, petroleum ether, diethyl ether, cyclopentane, cyclohexane, benzene, carbon tetrachloride, chloroform, methylcyclopentane, dimethylsulfide, 1,1-dichloroethane, 1,1,1-trichloroethane, perfluorohexane, perfluoroheptane, and 1-bromopropane.

117. A method of achieving lung volume reduction, comprising:
occluding a lumen of a bronchial tube of a lung with the bronchial occluder of claim 105 to prevent air flow to at least a region of the lung.

118. A method of achieving lung volume reduction, comprising:
dispensing a polymerizable monomer from the bronchial occlusion apparatus of claim 112 into a lumen of a bronchial tube of a lung to prevent air flow to at least a region of the lung.

119. The method of claim 118, wherein the pressurized gas of the bronchial occlusion apparatus causes the polymerizable monomer to expand upon dispensing the polymerizable monomer.

120. A kit for achieving lung volume reduction, comprising:
at least one bronchial occluder to substantially reduce or prevent air flow to at least a region of the lung without surgically removing said region of the lung; and
at least one intrabronchial applicator for said occluder.

121. The kit of claim 120, wherein said at least one bronchial occluder comprises a biocompatible composition comprising at least one monomer that forms a medically acceptable polymer.
